(19) United States
(12) Patent Application Publication
(10) Pub. No.: US 2024/0263199 A1
(43) Pub. Date: Aug. 8, 2024

(54) Method for Improving Production of L-lactic Acid by Saccharomyces Cerevisiae Based on Regulation and Control of Ethanol Metabolic Flux

(71) Applicant: Jiangnan University, Wuxi (CN)

(72) Inventors: Xueqin Lv, Wuxi (CN); Long Liu, Wuxi (CN); Jian Chen, Wuxi (CN); Guocheng Du, Wuxi (CN); Jianghua Li, Wuxi (CN); Yanfeng Liu, Wuxi (CN); Tiantian Liu, Wuxi (CN)

(21) Appl. No.: 18/604,597

(22) Filed: Mar. 14, 2024

(30) Foreign Application Priority Data
Mar. 17, 2023 (CN) 2023102595398

(51) Int. Cl. C12P 7/56; C12N 1/18; C12N 9/04; C12N 15/90; C12R 1/865

(52) U.S. Cl. CPC C12P 7/56; C12N 1/185; C12N 9/0006; C12N 15/905; C12Y 101/01027; C12Y 101/05005; C12R 2001/865

(57) ABSTRACT

The present disclosure discloses a method for improving production of L-lactic acid (L-LA) by Saccharomyces cerevisiae based on regulation and control of ethanol metabolic flux, and belongs to the technical field of microorganisms. According to the present disclosure, acid-resistant Saccharomyces cerevisiae TJG16 is used as a production strain, an ethanol dehydrogenase gene adhA derived from Bacillus subtilis is introduced to promote conversion of ethanol into acetaldehyde, and a lactate aldolase gene BAL derived from Brucella sp. is introduced to promote synthesis of lactic acid from the acetaldehyde. Moreover, an acetaldehyde dehydrogenase gene ALD6 is knocked out to prevent synthesis of acetic acid from the acetaldehyde, a transcriptional regulatory factor encoding gene GAL80 for regulating and controlling galactose is knocked out, and lactate dehydrogenase LDH is integrated, so that the L-LA is finally increased.

### REFERENCE TO SEQUENCE LISTING

The instant application contains a Sequence Listing in XML format as a file named “YGHY-2023-67-SEQ.xml”, created on Apr. 24, 2024, of 77,970 bytes in size, and which is hereby incorporated by reference in its entirety.

### TECHNICAL FIELD

The present disclosure relates to a method for improving production of L-lactic acid by Saccharomyces cerevisiae based on regulation and control of ethanol metabolic flux, and belongs to the technical field of microbial fermentation.

### BACKGROUND

L-lactic acid (L-LA, CH₃CHCOOH) is a natural organic acid, which has been widely used in food, medicine, cosmetics, tobacco, chemical engineering and other industries. Microbial fermentation has become a mainstream method for producing the L-LA due to advantages that a wide range of raw materials can be used, the production cost is low, high optical purity and high yield are achieved, and product safety is ensured. At present, Saccharomyces cerevisiae has been widely used in biosynthesis of many organic acids, such as L-malic acid, L-lactic acid and muconic acid, due to acid resistance and a clear genetic background. The biosynthesis of the L-LA can be achieved by introducing L-lactate dehydrogenase (L-LDH) into the Saccharomyces cerevisiae. On this basis, a number of metabolic regulatory strategies have been used in construction of cell factories for production of the L-LA by the Saccharomyces cerevisiae, including enhancing expression of a key enzyme L-LDH, weakening synthetic pathways of by-products and accelerating extracellular transport. For example, LDH derived from Lactobacillus Helveticus of Switzerland is used for replacing PDC1 by using an integrated expression strategy to construct a mutant strain carrying LDH with PDC1 deletion, and a titer of L-lactic acid is as high as 52.2 g/L. However, the Saccharomyces cerevisiae has problems of low heterologous gene expression efficiency and low yield, which have not been well solved yet.

In recent years, many studies have focused on the selection and isolation of acid-resistant Saccharomyces cerevisiae strains. For example, an acid-resistant (pH 4.2) strain (Saccharomyces cerevisiae BK01) was obtained by adaptive laboratory evolution (ALE) according to Jang et al., and the titer of L-LA of the strain was increased from 102 g/L to 119 g/L, which was increased by 17%. In our previous studies, a Saccharomyces cerevisiae mutant MTPfo-4 with tolerance to low pH (pH 2.4) was obtained by ALE (application No.: 202010631510.4). Moreover, a recombinant strain TJG16 was obtained by modification of a series of metabolic pathways (recorded in a patent document with a publication No.: CN114854612A), and the yield of L-LA reached 47.7 g/L. During production of the L-lactic acid by modification of the Saccharomyces cerevisiae, accumulation of the L-lactic acid and increase of the yield are achieved, and meanwhile, by-products are obviously decreased. However, the Saccharomyces cerevisiae has the characteristic of producing ethanol, and accumulation of the produced ethanol has a certain effect on growth of cells. Meanwhile, control of oxygen also has a certain effect on the production of the L-lactic acid, and lactic acid production strains need to be further developed and modified to improve the production of the L-lactic acid.

### SUMMARY

In order to solve the problems that ethanol produced by Saccharomyces cerevisiae affects the yield of L-lactic acid and the amount of oxygen is difficult to regulate and control, the present disclosure provides a method for further increasing the yield of the L-lactic acid in the Saccharomyces cerevisiae by introducing an ethanol dehydrogenase gene adhA derived from Bacillus subtilis to promote conversion of ethanol into acetaldehyde by way of a Cre-loxp technology, introducing a lactate aldolase gene BAL derived from Brucella sp. to promote synthesis of lactic acid from the acetaldehyde, and knocking out an acetaldehyde dehydrogenase gene ALD6 to prevent synthesis of acetic acid from the acetaldehyde.

The first purpose of the present disclosure is to provide recombinant Saccharomyces cerevisiae, where one or more of an ethanol dehydrogenase encoding gene adhA and a lactate aldolase encoding gene BAL are integrated on a genome of the recombinant Saccharomyces cerevisiae. In one embodiment, after the acetaldehyde dehydrogenase encoding gene ALD6 is knocked out, the gene adhA and the gene BAL are integrated at an ALD6 site.

In one embodiment, after the gene ALD6 is knocked out, the gene adhA and the gene BAL are integrated at the ALD6 site, and the gene adhA is integrated at a 1622b site.

In one embodiment, after the gene ALD6 is knocked out, the gene adhA and the gene BAL are integrated at the ALD6 site, the gene adhA is integrated at the 1622b site, and the gene BAL is integrated at a 1309a site.

In one embodiment, the recombinant Saccharomyces cerevisiae is obtained by further knocking out a transcriptional regulatory factor gene GAL80 for regulating and controlling galactose.

In one embodiment, after the gene GAL80 is knocked out, a lactate dehydrogenase encoding gene LDH is integrated at a GAL80 site with substituted simultaneously.

In one embodiment, at the ALD6 gene site, the gene adhA and the gene BAL are initially expressed by a galactose induced bidirectional promoter GAL1,10.

In one embodiment, at the 1622b site, the gene adhA is initially expressed by a promoter TEF1. In one embodiment, at the 1309a site, the gene BAL is initially expressed by a promoter BLA. In one embodiment, Saccharomyces cerevisiae TJG16 is used as a host cell, and the Saccharomyces cerevisiae TJG16 is recorded in a patent document with a publication No.: CN114854612A.

In one embodiment, the ethanol dehydrogenase adhA is derived from Bacillus subtilis and has a Gene ID of 938739, and the gene adhA has a nucleotide sequence as shown in SEQ ID NO: 1.

In one embodiment, the lactate aldolase BAL is derived from Brucella sp. and has a protein ID of EC 4.1.2.36, and the gene BAL has a nucleic acid sequence as shown in SEQ ID NO: 2.

In one embodiment, the acetaldehyde dehydrogenase encoding gene ALD6 has a nucleic acid sequence as set forth in SEQ ID NO: 75.

In one embodiment, the transcriptional regulatory factor encoding gene GAL80 for regulating and controlling galactose has a nucleic acid sequence as set forth in SEQ ID NO: 76.

In one embodiment, the bidirectional galactose induced promoter GAL1,10 has a nucleotide sequence as shown in SEQ ID NO: 3.

In one embodiment, the lactate dehydrogenase encoding gene LDH has a nucleotide sequence as shown in SEQ ID NO: 4.

In one embodiment, an upstream homologous arm at the 1309a site has a nucleotide sequence as shown in SEQ ID NO: 5, and a downstream homologous arm has a nucleotide sequence as shown in SEQ ID NO: 6; and an upstream homologous arm at the 1622b site has a nucleotide sequence as shown in SEQ ID NO: 7, and a downstream homologous arm has a nucleotide sequence as shown in SEQ ID NO: 8.

The second purpose of the present disclosure is to provide a method for producing L-lactic acid, where the method includes producing L-lactic acid by fermentation of the recombinant Saccharomyces cerevisiae.

In one embodiment, the recombinant Saccharomyces cerevisiae is inoculated into a fermentation system and cultured at 28° C.-35° C. and 200 rpm-220 rpm for 80 h-120 h.

In one embodiment, the recombinant Saccharomyces cerevisiae is cultured to reach an OD₆₀₀ value of 6±0.5, then inoculated into 15 L of a YPD culture medium at an amount of 8%-10% by volume percentage and cultured at 28° C.-35° C. and 200 rpm-220 rpm until a content of glucose in the system is less than 5 g/L, and then glucose is supplemented to maintain the content of glucose in the system at 20 g/L-25 g/L.

In one embodiment, aerobic fermentation is performed for 24 h before fermentation, and when the glucose is nearly consumed, anaerobic fermentation is performed by turning off oxygen.

In one embodiment, when the glucose is supplemented, CaCO₃ is supplemented to maintain the pH value of a fermentation solution at 4.5-5.

The third purpose of the present disclosure is to provide application of the recombinant Saccharomyces cerevisiae in preparation of L-lactic acid, L-lactic acid derivatives, products containing L-lactic acid and products containing L-lactic acid derivatives.

The present disclosure has the following beneficial effects.

According to the present disclosure, acid-resistant Saccharomyces cerevisiae TJG16 is used as a production strain, which has acid resistance of Saccharomyces cerevisiae in fermentation to produce organic acids, thereby greatly increasing the yield of L-lactic acid. The Saccharomyces cerevisiae TJG16 is modified. Specifically, an ethanol dehydrogenase gene adhA derived from Bacillus subtilis is introduced to promote conversion of ethanol into acetaldehyde, and a lactate aldolase gene BAL derived from Brucella sp. is introduced to promote synthesis of lactic acid from the acetaldehyde. Moreover, an acetaldehyde dehydrogenase gene ALD6 is knocked out to prevent synthesis of acetic acid from the acetaldehyde, a transcriptional regulatory factor encoding gene GAL80 for regulating and controlling galactose is knocked out, and a lactate dehydrogenase gene LDH is integrated, so that the yield of L-LA is obviously increased eventually. The yield is increased from initial 47.7 g/L to 50.5 g/L-192.3 g/L.

### BRIEF DESCRIPTION OF FIGURES

FIG. 1 shows high performance liquid chromatography of an L-LA standard product; and

FIG. 2 shows high performance liquid chromatography results of L-LA obtained by fermentation of a Saccharomyces cerevisiae strain TJG20.

### DETAILED DESCRIPTION

The present disclosure is further explained in combination with specific examples below to enable the present disclosure better understood and implemented by persons skilled in the art, but the examples provided are not used as limitations of the present disclosure.

### (I) Culture Medium:

An LEU-plate was obtained by adding glucose in combination with histidine (HIS), uracil and tryptophan on the basis of an amino acid-free yeast nitrogen base (YNB) culture medium, and was used for screening genetically modified bacteria with a tag LEU.

An HIS-plate was obtained by adding glucose in combination with leucine (LEU), uracil and tryptophan on the basis of an amino acid-free yeast nitrogen base (YNB) culture medium, and was used for screening genetically modified bacteria with a tag HIS.

A YPD liquid culture medium includes 20 g/L of peptone, 10 g/L of yeast powder and 20 g/L of glucose.

(II) Preparation of Competent Cells of Saccharomyces cerevisiae:
(1) Fresh recombinant Saccharomyces cerevisiae was selected from a YPD plate, monocloned into 10 ml of the YPD liquid culture medium and then cultured overnight at 30° C. and 250 rpm.
(2) An overnight culture was determined to have an OD₆₀₀ value of 3.0-5.0.
(3) 10 ml of the YPD overnight culture was diluted to reach an OD₆₀₀ value of 0.2-0.4.
(4) The culture was continuously cultured in a shaker at 28° C.−30° C. for 3 h-6 h to reach an OD₆₀₀ value of 0.6-1.0.
(5) Centrifugation was performed at 1,500 g at room temperature for 5 min, yeast cells were collected, and a supernatant was discarded.
(6) The yeast cells were washed with 10 ml of an eluting agent and then centrifuged at 1,500 g at room temperature for 5 min, the cells were collected, and a supernatant was discarded.
(7) The yeast cells were resuspended with 1 mL of TE/LiAc and then separately packaged in tubes at 50 μL/tube.
   (III) Transformation of Saccharomyces cerevisiae:
(1) 50 μl of the competent cells were taken, added into 2 μl of each plasmid to be transferred and then uniformly mixed.
(2) 500 μl of a transformation solution (PEG/LiAc, dimethyl sulfoxide) was added, and a tube wall was flicked to mix the solution uniformly.
(3) Treatment was performed in a water bath at 30° C. for 1 h, and the tube wall was flicked every 15 min to mix the solution uniformly.
(4) 1 mL of the YPD culture solution was added and cultured in the shaker at 30° ° C. for 1 h.
(5) Centrifugation was performed at 3,500 g for 5 min, a precipitate was remained, and a supernatant was discarded.
(6) The precipitate was resuspended with 150 μL of TE and coated on the corresponding SD plate; and the plate was inverted for culture at 30° C.

### (IV) Detection of L-Lactic Acid:

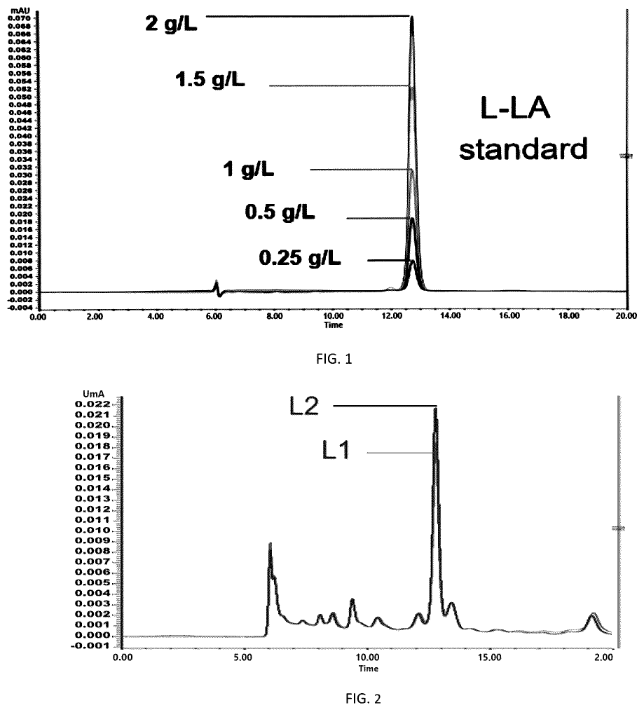

L-LA in the Saccharomyces cerevisiae was detected by high performance liquid chromatography. 1 ml of a Saccharomyces cerevisiae bacterial solution fermented for 112 h was added into 0.5 mm glass beads and crushed for 20 min by using a high-speed homogenizing and crushing instrument, a crushed mixture was taken out and centrifuged to obtain a supernatant, and the supernatant was diluted for 10 times, filtered with a 0.55 μm aqueous membrane and then analyzed by high performance liquid chromatography. 0.5 mM dilute sulfuric acid was used as a mobile phase at a flow rate of 0.6 mL/min. An ultraviolet detector with a detection wavelength of 210 nm and a detection temperature of 50° C. was used as a detector. The high performance liquid chromatography of an L-LA standard product is shown in FIG. 1.
   (V) A Saccharomyces cerevisiae Strain TJG16 Used in the Present Application was Published in a patent document with a publication No. CN114854612A.

Primers used in examples are shown in Table 1.

TABLE 1

| Primer | | | |
|---|---|---|---|
| name | Sequence | | Effect |
| ALD6-U-F | atgactaagctacactttgacactgctg | SEQ ID NO: | Bidirectionally |
| | | 9 | expressing |
| | | | two genes |
| | | | of adhA and |
| | | | BAL by a |
| | | | promoter |
| | | | GAL1, 10, |
| | | | and |
| | | | knocking |
| | | | out a gene |
| | | | ALD6 |
| ALD6-U-R | cgtaatcatggtcatagctgtttcctggaccacagacaccg | SEQ ID NO: | |
| | attggc | 10 | |
| LEU-A-F | gagccaatcggtgtctgtggtccaggaaacagctatgacc | SEQ ID NO: | |
| | atgattacg | 11 | |
| LEU-A-R | gctgtatagctcatatctttccctttaaaacgacggccagtg | SEQ ID NO: | |
| | cca | 12 | |
| TDH3-A-F | ggcactggccgtcgttttaaagggaaagatatgagctata | SEQ ID NO: | |
| | cagcgg | 13 | |
| TDH3-A-R | gtgattgatatttccacattgtaaagtgaatttactttaaatc | SEQ ID NO: | |
| | ttgcatttaaataaattttctttttatagc | 14 | |
| ADHA-A- | caagatttaaagtaaattcactttacaatgtggaaatatca | SEQ ID NO: | |
| F | atcacaaatcgataacg | 15 | |
| ADHA-A- | gtaagaatttttgaaaattcaatataaatgtgtaatcaaca | SEQ ID NO: | |
| R | tcaaacccgtgtattaag | 16 | |
| GAL-A-F | gttgattacacatttatattgaattttcaaaaattcttactttt | SEQ ID NO: | |
| | tttttggatggacg | 17 | |
| GAL-A-R | gaataatttgcattatagttttttctccttgacgttaaagtat | SEQ ID NO: | |
| | agaggtatattaacaat | 18 | |
| BAL-A-F | cgtcaaggagaaaaaactataatgcaaattattcatactat | SEQ ID NO: | |
| | tgaagaattgagacaagc | 19 | |
| BAL-A-R | gtaagcgtgacataactaattacatgattaagcagctcttt | SEQ ID NO: | |
| | cttgagtaatttgtggag | 20 | |
| CYC1-A-F | ttactcaagaaagagctgcttaatcatgtaattagttatgtc | SEQ ID NO: | |
| | acgcttacattcacg | 21 | |
| CYC1-A-R | gtcaaatggattaccaactttgatggccgcaaattaaagcc | SEQ ID NO: | |
| | ttcga | 22 | |
| ALD6-D-F | ctcgaaggctttaatttgcggccatcaaagttggtaatccat | SEQ ID NO: | |
| | ttgacaaggctaa | 23 | |
| ALD6-D-R | ttacaacttaattctgacagcttttacttcagtgtatg | SEQ ID NO: | |
| | | 24 | |
| A-Y-F | gttaccattgcaatcaactgtctaagagatg | SEQ ID NO: | Validation |
| | | 25 | |
| A-Y-R | caagaacgaattccctacattgaaggt | SEQ ID NO: | |
| | | 26 | |
| A-Y1-F | caagtcgaccttggcactgg | SEQ ID NO: | |
| | | 27 | |
| A-Y1-R | gccatgtaatatgattattaaacttctttgcgtcc | SEQ ID NO: | |
| | | 28 | |
| A-Y2-F | gttaatatacctctatactttaacgtcaaggagaaaaaact | SEQ ID NO: | |
| | ataatg | 29 | |
| A-Y2-R | gaaaacggttggtctgatgaagtaacc | SEQ ID NO: | |
| | | 30 | |
| 1622b-U- | atgtctctcctgcatcactaaatgtgtt | SEQ ID NO: | Expressing |
| F | | 31 | adhA at a |
| | | | 1622b site |
| | | | of a |
| | | | genome |
| 1622b-U- | gtaatcatggtcatagctgtttcctgtcagcaaagtcaaga | SEQ ID NO: | |
| R | atccaaattctggc | 32 | |
| LEU-A1-F | gaatttggattcttgactttgctgacaggaaacagctatga | SEQ ID NO: | |
| | ccatgattacg | 33 | |
| LEU-A1-R | ggagtagaaacattttgaagctattaaaacgacggccagt | SEQ ID NO: | |
| | gccaa | 34 | |
| TEF1-A-F | gcactggccgtcgttttaatagcttcaaaatgtttctactcct | SEQ ID NO: | |
| | tttttactcttcc | 35 | |
| TEF1-A-R | gtttgatgttgattacacataaacttagattagattgctatg | SEQ ID NO: | |
| | ctttctttctaatgag | 36 | |
| ADHA-F | catagcaatctaatctaagtttatgtgtaatcaacatcaaa | SEQ ID NO: | |
| | cccgtgtattaag | 37 | |
| ADHA-R | gtgacataactaattacatgattacaatgtggaaatatcaa | SEQ ID NO: | |
| | tcacaaatcgataacg | 38 | |
| CYC1-A-F | gtgattgatatttccacattgtaatcatgtaattagttatgtc | SEQ ID NO: | |
| | acgcttacattcacg | 39 | |
| CYC1-A1- | gagtgtttatgggtgtcatggccgcaaattaaagccttcga | SEQ ID NO: | |
| R | | 40 | |
| 1622b-D- | gaaggctttaatttgcggccatgacacccataaacactccc | SEQ ID NO: | |
| F | gac | 41 | |
| 1622b-D- | acaataccatataccaacggcaatattcagc | SEQ ID NO: | |
| R | | 42 | |
| Y1-1622- | gatttccttccaggatgtatcttagacgaac | SEQ ID NO: | Validation |
| U | | 43 | |
| Y1-1622- | gaacaatacaccgttccagaagtgc | SEQ ID NO: | |
| D | | 44 | |
| Y2-1622- | ctcgatgtagatgcctatttatcaatgcttc | SEQ ID NO: | |
| U | | 45 | |
| Y2-1622- | gtagctttaggattcatgaatattgtcatctcattattcg | SEQ ID NO: | |
| D | | 46 | |
| 1309-U-F | cagaaaaacagatgtgcccaaatccac | SEQ ID NO: | Expressing |
| | | 47 | BAL at a |
| | | | 1309a site |
| | | | of a |
| | | | genome |
| 1309-U-R | catggtcatagctgtttcctggatcctaaactgcgtcatagt | SEQ ID NO: | |
| | aagtttctttg | 48 | |
| HIS-B-F | cttactatgacgcagtttaggatccaggaaacagctatgac | SEQ ID NO: | |
| | catgattacg | 49 | |
| HIS-B-R | gcgacacggaaatgttgaatactcattaaaacgacggcca | SEQ ID NO: | |
| | gtgcca | 50 | |
| BLA-B-F | cttggcactggccgtcgttttaatgagtattcaacatttccgt | SEQ ID NO: | |
| | gtcgc | 51 | |
| BLA-B-R | caatagtatgaataatttgcatccaatgcttaatcagtgag | SEQ ID NO: | |
| | gcacc | 52 | |
| BAL-F | cctcactgattaagcattggatgcaaattattcatactattg | SEQ ID NO: | |
| | aagaattgagacaagc | 53 | |
| BAL-R | gcaagatttaaagtaaattcactttaagcagctctttcttga | SEQ ID NO: | |
| | gtaatttgtgg | 54 | |
| TDH3-B-F | gagctgcttaaagtgaatttactttaaatcttgcatttaaat | SEQ ID NO: | |
| | aaattttctttttatagc | 55 | |
| TDH3-B-R | gctttacgatggagtagtagacctaagggaaagatatgag | SEQ ID NO: | |
| | ctatacagcgg | 56 | |
| 1309-D-F | cgctgtatagctcatatctttcccttaggtctactactccatc | SEQ ID NO: | |
| | gtaaagcc | 57 | |
| 1309-D-R | tgaggaatttacaataaggtggttcctttagttataaattg | SEQ ID NO: | |
| | | 58 | |
| Y1-BAL-F | ctatttataaacgtcactaactagaaatacgggatatcaac | SEQ ID NO: | Validation |
| | tacta | 59 | |
| Y1-BAL-R | caaaaacaggaaggcaaaatgccg | SEQ ID NO: | |
| | | 60 | |
| Y2-BAL-F | gtaagccccccgtatcgtagttatc | SEQ ID NO: | |
| | | 61 | |
| Y2-BAL-R | gagaactgaaatttccataccctttaaccct | SEQ ID NO: | |
| | | 62 | |
| GAL80-U- | caattcaagatacagaacctcctccagatg | SEQ ID NO: | Knocking |
| F | | 63 | out a gene |
| | | | GAL80 to |
| | | | relieve |
| | | | galactose |
| | | | induction |
| GAL80-U- | cgtaatcatggtcatagctgtttcctggtggaaagaacggg | SEQ ID NO: | |
| R | aaaccaactatc | 64 | |
| G-HIS-F | gatagttggtttcccgttctttccaccaggaaacagctatga | SEQ ID NO: | |
| | ccatgattacgc | 65 | |
| G-HIS-R | gaaacattttgaagctattaaaacgacggccagtgcca | SEQ ID NO: | |
| | | 66 | |
| G-LLDH-F | gcactggccgtcgttttaatagcttcaaaatgtttctactcct | SEQ ID NO: | |
| | tttttactcttc | 67 | |
| G-LLDH-R | cactgggggccaagcacagggggccgcaaattaaagcct | SEQ ID NO: | |
| | tcgag | 68 | |
| GAL80-D- | ctcgaaggctttaatttgcggccccctgtgcttggcccc | SEQ ID NO: | |
| F | | 69 | |
| GAL80-D- | gccgatttgtattagtatccaaattatgattccatg | SEQ ID NO: | |
| R | | 70 | |
| Y1-G80-F | cttccatagagagaaggagcaagcaac | SEQ ID NO: | Validation |
| | | 71 | |
| Y1-G80-R | gtactagaggaggccaagagtaatagaaaaag | SEQ ID NO: | |
| | | 72 | |
| Y2-G80-F | cacttattacggaattggaatgagcacag | SEQ ID NO: | |
| | | 73 | |
| Y2-G80-R | cagcaaaacatgcttattgtaattgggc | SEQ ID NO: | |
| | | 74 | |

### Example 1: Construction of a Recombinant Saccharomyces cerevisiae Strain TJG17

A gene adhA derived from Bacillus subtilis (with a nucleotide sequence as shown in SEQ ID NO: 1) and a gene BAL derived from Brucella sp. (with a nucleotide sequence as shown in SEQ ID NO: 2) were integrated at an ALD6 site of Saccharomyces cerevisiae TJG16 to achieve overexpression of the gene adhA and the gene BAL.

The Saccharomyces cerevisiae strain TJG16 was prepared into yeast competent cells.

With a genome of Saccharomyces cerevisiae S288C as a template, primers ALD6-U-F/R, LEU-A-F/R, TDH3-A-F/R, ADHA-A-F/R, GAL-A-F/R, BAL-A-F/R, CYC1-A-F/R and ALD6-D-F/R (Table 1) were used for amplification to obtain 8 recombinant fragments: ALD6-U, tag LEU, terminator TDH3, adhA, GAL1,10, BAL, terminator CYC1 and ALD6-D, respectively. The obtained 8 recombinant fragments were co-transformed into the competent cells of Saccharomyces cerevisiae TJG16, coated on an LEU-plate and then cultured at 30° C. for 2-3 days until single bacterial colonies grew. Primers A-Y-F/R, A-Y1-F/R and A-Y2-F/R were used for validation to verify a correct strain, namely a positive transformer with double expression of two genes adhA and BAL, which was named as strain TJG17.

### Example 2: Construction of Recombinant Saccharomyces cerevisiae Strains TJG18 to TJG20

(a) Construction of a Recombinant Saccharomyces cerevisiae Strain TJG18

A gene adhA derived from Bacillus subtilis (with a nucleotide sequence as shown in SEQ ID NO: 1) was integrated at a 1622b site of Saccharomyces cerevisiae TJG17 to achieve multi-copy expression of the gene adhA and promote synthesis of acetaldehyde from ethanol.

The strain TJG17 constructed in Example 1 was prepared into yeast competent cells.

With a genome of a Saccharomyces cerevisiae engineering strain S288C as a template, primers 1622b-U-F and 1622b-U-R were used for amplification to obtain a gene fragment 1622b-U. Primers LEU-A1-F and LEU-A1-R were used for amplification to obtain a tag gene fragment LEU, and primers TEF1-A-F and TEF1-A-R were used for amplification to obtain a promoter TEF1. Primers ADHA-F and ADHA-R were used for amplification to obtain adhA. Primers CYC1-A1-F and CYC1-A1-R were used for amplification of the gene fragment to obtain a terminator CYC1. Primers 1622b-D-F and 1622b-D-R were used for amplification to obtain a gene fragment 1622b-D. The gene fragments 1622b-U, LEU, TEF1, adhA, CYC1 and 1622b-D were transferred into the competent cells of Saccharomyces cerevisiae TJG17 by chemical transformation, coated on an LEU-plate and then cultured at 30° C. for 2-3 days until single bacterial colonies grew. Primers Y1-1622-U/D and Y2-1622-U/D were used for performing PCR validation on the bacterial colonies, and a correctly verified strain was named as TJG18.

(b) Construction of a Recombinant Saccharomyces cerevisiae Strain TJG19

A gene BAL derived from Brucella sp. (with a nucleotide sequence as shown in SEQ ID NO: 2) was integrated at a 1309a site of Saccharomyces cerevisiae TJG18 to achieve multi-copy expression of the gene BAL and promote synthesis of lactic acid from acetaldehyde.

The strain TJG18 constructed in step (a) was prepared into yeast competent cells.

With a genome of a Saccharomyces cerevisiae engineering strain S288C as a template, primers 1309-U-F and 1309-U-R were used for amplification to obtain a gene fragment 1309a-U. Primers HIS-B-F and HIS-B-R were used for amplification to obtain a tag gene fragment HIS, and primers BLA-B-F and BLA-B-R were used for amplification to obtain a promoter BLA. Primers BAL-F and BAL-R were used for amplification to obtain BAL. Primers TDH3-B-F and TDH3-B-R were used for amplification of the gene fragment to obtain a terminator TDH3. Primers 1309-D-F and 1309-D-R were used for amplification to obtain a gene fragment 1309-D. The gene fragments 1309a-U, HIS, BLA, BAL, TDH3 and 1309-D were transferred into the competent cells of Saccharomyces cerevisiae TJG18 by chemical transformation, coated on an HIS-plate and then cultured at 30° C. for 2-3 days until single bacterial colonies grew. Primers Y1-BAL-F/R and Y2-BAL-F/R were used for performing PCR validation on the bacterial colonies, and a finally obtained strain was named as TJG19.

(c) Construction of a Recombinant Saccharomyces cerevisiae Strain TJG20

A lactate dehydrogenase gene LDH was integrated at a GAL80 site of Saccharomyces cerevisiae TJG19 to achieve knockout of a gene GAL80, and a synthetic pathway of L-lactic acid was initiated without adding galactose.

Similar to the steps in step (b), with a genome of a Saccharomyces cerevisiae engineering strain S288C as a template, primers GAL80-U-F and GAL80-U-R were used for amplification to obtain a gene fragment GAL80-U. Primers GAL80-D-F and GAL80-D-R were used for amplification to obtain a gene fragment GAL80-D. Primers G-HIS-F and G-HIS-R were used for amplification to obtain a tag gene fragment HIS. Primers G-LLDH-F and G-LLDH-R were used for amplification to obtain a gene fragment LLDH (including a promoter TEF1, lactate dehydrogenase LDH and a terminator CYC1). The gene fragments GAL80-U, LLDH and HIS were transferred into the competent cells of Saccharomyces cerevisiae TJG19 by chemical transformation, coated on an HIS-plate and then cultured at 30° C. for 2-3 days until single bacterial colonies grew. Primers Y1-G80-FR and Y2-G80-F/R were used for performing PCR validation on the bacterial colonies, and a finally obtained strain was named as TJG20.

### Example 3: Production of L-Lactic Acid by Fermentation of Recombinant Saccharomyces cerevisiae

The single bacterial colonies of the Saccharomyces cerevisiae strains TJG17 to TJG20 constructed in Example 1 and Example 2 that were selected from solid YPD plates were inoculated into 2 mL of a YPD liquid culture medium for culture at 30° C. and 220 rpm for 18-24 h, respectively, and then inoculated into a 30 L fermentation tank containing 15 L of a YPD liquid culture medium at a volume ratio of 10% for culture at 30° C. and 220 rpm when the OD₆₀₀ value of the fermentation strains reached about 6. The single bacterial colonies were subjected to aerobic fermentation for 24 h before fermentation, and then subjected to anaerobic fermentation by turning off oxygen when glucose was nearly consumed. When a content of glucose was less than 5 g/L during the fermentation, glucose was added to supplement a carbon source so as to maintain the content of glucose at 20 g/L-25 g/L. When the glucose was supplemented, CaCO₃ was supplemented to maintain the pH value of a fermentation solution at 4.5-5.

After the fermentation was performed for a total of 112 h, centrifugation was performed to obtain a precipitate, and a supernatant was discarded. The precipitate was resuspended with 10 mL of sterile water, added into 0.5 mm glass beads and crushed for 20 min by using a high-speed homogenizing and crushing instrument, and a crushed mixture was taken out, filtered with a 0.55 μm membrane and then analyzed by high performance liquid chromatography. Dilute sulfuric acid was used as a mobile phase, and an ultraviolet detector with a detection wavelength of 210 nm and a detection temperature of 50° C. was used as a detector.

According to analysis by high performance liquid chromatography, on the basis of TJG16, the L-LA yields of the successfully constructed high-yield lactic acid strains TJG17 to TJG20 are 50.5 g/L, 72.7 g/L, 119.0 g/L and 192.3 g/L, respectively (FIG. 2).

The strain TJG16 was fermented by the above method to produce L-lactic acid, and the yield of L-lactic acid was determined as 47.7 g/L.

Although the present disclosure has been disclosed above as preferred examples, the examples are not intended to limit the present disclosure, and various changes and modifications can be made by any person familiar with the art without departing from the spirit and scope of the present disclosure. Therefore, the scope of protection of the present disclosure shall be defined by the claims.

What is claimed is:

1. Recombinant Saccharomyces cerevisiae, wherein Saccharomyces cerevisiae TJG16 is used as a host cell, and after an acetaldehyde dehydrogenase encoding gene ALD6 is knocked out at an ALD6 site, a gene adhA and a gene BAL are integrated at the ALD6 site;
alternatively, after the gene ALD6 is knocked out, the gene adhA and the gene BAL are integrated at the ALD6 site, and the gene adhA is integrated at a 1622b site;
alternatively, after the gene ALD6 is knocked out, the gene adhA and the gene BAL are integrated at the ALD6 site, the gene adhA is integrated at the 1622b site, and the gene BAL is integrated at a 1309a site; and
the gene adhA has a nucleotide sequence as set forth in SEQ ID NO: 1; the gene BAL has a nucleic acid sequence as set forth in SEQ ID NO: 2; and the gene ALD6 has a nucleic acid sequence as set forth in SEQ ID NO: 75.

2. The recombinant Saccharomyces cerevisiae according to claim 1, wherein the recombinant Saccharomyces cerevisiae is obtained by further knocking out a transcriptional regulatory factor gene GAL80 for regulating and controlling galactose, and the gene GAL80 has a nucleic acid sequence as set forth in SEQ ID NO: 76.

3. The recombinant Saccharomyces cerevisiae according to claim 2, wherein after the gene GAL80 is knocked out, a lactate dehydrogenase encoding gene LDH is integrated at a GAL80 site, and the lactate dehydrogenase encoding gene LDH has a nucleotide sequence as set forth in SEQ ID NO: 4.

4. A method for producing L-lactic acid, comprising producing L-lactic acid by fermentation of the recombinant Saccharomyces cerevisiae according to claim 1.

5. The method according to claim 4, comprising inoculating the recombinant Saccharomyces cerevisiae into a fermentation system, culturing to reach an OD₆₀₀ value of 6±0.5, then inoculating into a YPD culture medium at an amount of 8%-10% by volume percentage and culturing at 28° C.-35° C. and 200 rpm-220 rpm until a content of glucose in the system is less than 5 g/L, and then glucose is supplemented to maintain the content of glucose in the system at 20 g/L-25 g/L.


SEQUENCE LISTING (76 sequences)

SEQ ID NO: 1 - DNA, 1049 bp, synthetic construct
  source 1..1049: mol_type = other DNA
atgtgtaatcaacatcaaacccgtgtattaagcgtgtcacatgcaaaagccaaatttgaacaaacgacgattgagcgaagaggattacggccgcacgatgtcttaatcgatattaaattcagcggcatttccattcagacattcatagcgcttttgatgaatggggcggtggcatcttcccaatggtgcctggacatgaaatagccggtgttgtaacagccgttggaacaaaagtcaccaagttggctgtcggggaccgcgttggtgtcggctgttttgttgactcttgcggagaatgcgaatactgtctcaacgcagaggaacaattctgcacgaagggtgttgttcaaacgtataactcagtagactacgacggaaaccccacttatggcgggtacagccaaaaaatcgttgtcactgacagatttgttgttcgcataccagatcgtttggaaatggatgttgccagtccgctgttgtgtgcaggtatcacaacgtattccccgctgaaacactggaatgttggccctggtaaaaaggttgcgattgtaggcgtaggtggccttggacatttggcaattcaatttgcacatgcgatgggtgcagaagtaaccgtcctgagccgttccatgaataagaaagaggaagctctggaactcggtgcaaatcattactttgcaacaagtgacccagccacattcactgcattggccgggcgttttgacgtgatcttaaatacggtatcggcaaatctcgatgtagatgcctatttatcaatgcttcgtatagacgggacacttgtaagcgttggagctcctgccaagccggacacatacagtgtgttttccctaatcatggggcgccgcagcattgcaggttctctagtcggcggaattcaagaaactcaagagatgctggatttcgcagctgagcatggcatcgaacctaaaatcgaagtcatcggagctgaccaagtggacgaggcatacgaacgtatcctccgaagcgatgtccgttatcgatttgtgattgatatttccacattgtaa

SEQ ID NO: 2 - DNA, 882 bp, synthetic construct
  source 1..882: mol_type = other DNA
atgcaaattattcatactattgaagaattgagacaagctttggctcctgctagacaacaaggtaaaaaaattggttttgttccaactatgggttatttgcataaaggtcatttggaattggttagaagagctagagttgaaaatgatgttactttggtttctatttttgttaatccattgcaatttggtgctaatgaagatttgggtagatatccaagagatttggaaagagatgctggtttgttgcatgatgctcaagttgattatttgtttgctccaactgtttctgatatgtatccaagaccaatgcaaactgttgttgatgttccaccattgggtaatcaaatggaaggtgaagctagacctggtcattttgctggtgttgctactgttgtttctaaattgtttaatattgttggtcctgatgctgcttattttggtgaaaaagattttcaacaattggttattattagaagaatggttgatgatatggctattcctgttagaattgttggtgttgaaactgttagagaagatgatggtttggcttgttcttctagaaatgtttatttgactcctgaacaaagaagagctgctattattgttccacaagctttggatgaagctgatagattgtatagatctggtatggatgatcctgatgctttggaagctgctattagaacttttattggtagacaaccattggctgttcctgaagttattgctattagagatcctgaaactttggaaagattgcctgctttgcaaggtagaccaattttggttgctttgtttgttagagttggtgctactagattgttggataatagagttattggtcatgctgctccacaaattactcaagaaagagctgcttaa

SEQ ID NO: 3 - DNA, 668 bp, synthetic construct
  source 1..668: mol_type = other DNA
ttatattgaattttcaaaaattcttactttttttttggatggacgcaaagaagtttaataatcatattacatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 4 - DNA, 945 bp, synthetic construct
  source 1..945: mol_type = other DNA
atgaaaattacaagtagaaaagtagtcgtgattggaacaggatttgtcggaacaagtattgcttattcaatgattaatcaagggcttgtcaacgagttagttttgatagatgtcaatcaagataaagctgagggtgaggctttagatttattggatggcatatcttgggctcaagaaaatgttattgtaagagctggaaattacaaggattgtgaaaatgcagatattgtcgtgattacagctggtgtgaaccaaaaacccggacaatcacgtttagatttggtcaatacaaatgctaaaattatgcgctcaatcgttactcaagttatggattcaggttttgatggtatttttgtaattgcttctaatcctgtggatatccttacctatgtcgcttgggaaacttctggtcttgaccaatctagaattgtcggaactggtacaactttggataccacacgttttagaaaagaattggccacaaaactagaaattgaccctcggagtgttcacggatatattattggtgagcatggcgattctgaagttgcagtttggtcacacactacgataggtggaaaacctattcttgaatttattgttaaaaataaaaaaattggtcttgaagatttgtctaatttgtcaaataaggttaaaaatgctgcttatgaaattattgataaaaaacaagccacttattacggaattggaatgagcacagctagaattgtcaaagccatattaaataatgaacaagtcattctaccagtttctgcctatttgcgtggagaatatggtcaagaaggggtctttactggtgttccatctgtcgtaaatcaaaatggtgtcagagaaattattgaactcaatattgatgcttatgaaatgaaacaatttgaaaagtctgtcagtcaacttaaagaagtcatagaatctattaaataa

SEQ ID NO: 5 - DNA, 545 bp, synthetic construct
  source 1..545: mol_type = other DNA
cagaaaaacagatgtgcccaaatccacgatgaggttcctcattcccgttgttgatcttgtagtctgggtttttttcagtaccccaattgtgagttagttctaagacaccgtgtgcgctaaaaacatcaggctctccattcttatttttgggtatgtcgtcttttggaaagcttaaaaagtacaagctaaattttgcttcttcaaaatcctttctgcttaatagcttcataccgaagtgttcggtgtagaacttaacggtccttgctggatccttgactcttaaacatgtgtgattaagcagaagggttggatcattcgaggctttctcaatctgaattggatagcgtgtactatcagtggacattgttagtttgtagctggtggcgtatctggtctgaagtgtcaaaaaaaaggcatggaaatgttattccgattttttttctccctatttataaacgtcactaactagaaatacgggatatcaactactacgagagagggacattctagtatcaaagaaacttactatgacgcagtttaggatc

SEQ ID NO: 6 - DNA, 564 bp, synthetic construct
  source 1..564: mol_type = other DNA
aggtctactactccatcgtaaagccccttgtttattcagtgtccagatcatatgcgggatgtgtattttgtgctgagtggaaaaataagatcatatgctcaagaataaagggttaaagggtatggaaatttcagttctccctggctcaagaacattgtcagtattcctataactgaagctgagtggtactaatgcaccgagatgcaaagtccgaaccacccccacccccctctttacctattttttatattttgtgataaagggataactcgtcttgccttcactgcgcttcgattctactttagctaaactaagcgttactttgtttgagaatctctcagccgttaaatcaacgtacttgtatactaaacagtatattcacgtgataactttgccttacacctatgatatagataaatatatacatatatatgagtgtgtgtgcattgcgtcactaaactatgtttaatatgacagaaggttttcgtttaattttgccgctacccgaaaattttggcagttgcaatttataactaaaggaaccaccttattgtaaattcctca

SEQ ID NO: 7 - DNA, 511 bp, synthetic construct
  source 1..511: mol_type = other DNA
atgtctctcctgcatcactaaatgtgttgctagtattattttcgttgttatttgcttgcgctctttccacactagcagctacacctgctttgaataatcgccttcttactttgtcatttatagatacattgacaaggtaaataaagacattgcgcctcttgtaactagtgataatttcctccagcacttgggcggctgaagaatctattgaggtcatcccacctaaatcgaaaatgacatattttatggaatccttcgagcgaagtgatttccttccaggatgtatcttagacgaaccgtacctctctattctgtcaagtctctgttttaaatcttcgctgtttgtaaatgtgagaggctcaggtattctaacaatcatgcatccctcgatttcttctgtgccctcaacatccagagagttccttttcatgttcatcatgtagtcgtcaagattcgtaaagttcgacgttcccgctaccctagccagaatttggattcttgactttgctga

SEQ ID NO: 8 - DNA, 516 bp, synthetic construct
  source 1..516: mol_type = other DNA
atgacacccataaacactcccgacattacgctttgtgctcctgatagggcgttgatcttggatcttccatatccgccgaatgcgggaagagcaccgaataatgagatgacaatattcatgaatcctaaagctactagttctctattagaggaaacggtaagattgtaagttgttccaagagatttcgacgcggtcgtagattcaaaaaaacccaacattgccactattagagatgcactaaaaagatctggtattagcttacgtctaggacgagttaaagggtttttcaattcatcaaaattgtccatgctaaagtcgccaatgatagaaataccatatctgtgttttaaattaaatttcatggaaataagaatagtaacaattaccaccaataatatatctggaaaaaatattgcgctcttgtgatatttcattaatttcctctttagtagacgcgttaaaaagaggacaatcaagcaacatccgctgaatattgccgttggtatatggtattgt

SEQ ID NO: 9 - DNA, 28 bp, synthetic construct
  source 1..28: mol_type = other DNA
atgactaagctacactttgacactgctg

SEQ ID NO: 10 - DNA, 47 bp, synthetic construct
  source 1..47: mol_type = other DNA
cgtaatcatggtcatagctgtttcctggaccacagacaccgattggc

SEQ ID NO: 11 - DNA, 49 bp, synthetic construct
  source 1..49: mol_type = other DNA
gagccaatcggtgtctgtggtccaggaaacagctatgaccatgattacg

SEQ ID NO: 12 - DNA, 45 bp, synthetic construct
  source 1..45: mol_type = other DNA
gctgtatagctcatatctttccctttaaaacgacggccagtgcca

SEQ ID NO: 13 - DNA, 46 bp, synthetic construct
  source 1..46: mol_type = other DNA
ggcactggccgtcgttttaaagggaaagatatgagctatacagcgg

SEQ ID NO: 14 - DNA, 73 bp, synthetic construct
  source 1..73: mol_type = other DNA
gtgattgatatttccacattgtaaagtgaatttactttaaatcttgcatttaaataaattttctttttatagc

SEQ ID NO: 15 - DNA, 58 bp, synthetic construct
  source 1..58: mol_type = other DNA
caagatttaaagtaaattcactttacaatgtggaaatatcaatcacaaatcgataacg

SEQ ID NO: 16 - DNA, 59 bp, synthetic construct
  source 1..59: mol_type = other DNA
gtaagaatttttgaaaattcaatataaatgtgtaatcaacatcaaacccgtgtattaag

SEQ ID NO: 17 - DNA, 58 bp, synthetic construct
  source 1..58: mol_type = other DNA
gttgattacacatttatattgaattttcaaaaattcttactttttttttggatggacg

SEQ ID NO: 18 - DNA, 60 bp, synthetic construct
  source 1..60: mol_type = other DNA
gaataatttgcattatagttttttctccttgacgttaaagtatagaggtatattaacaat

SEQ ID NO: 19 - DNA, 59 bp, synthetic construct
  source 1..59: mol_type = other DNA
cgtcaaggagaaaaaactataatgcaaattattcatactattgaagaattgagacaagc

SEQ ID NO: 20 - DNA, 59 bp, synthetic construct
  source 1..59: mol_type = other DNA
gtaagcgtgacataactaattacatgattaagcagctctttcttgagtaatttgtggag

SEQ ID NO: 21 - DNA, 57 bp, synthetic construct
  source 1..57: mol_type = other DNA
ttactcaagaaagagctgcttaatcatgtaattagttatgtcacgcttacattcacg

SEQ ID NO: 22 - DNA, 46 bp, synthetic construct
  source 1..46: mol_type = other DNA
gtcaaatggattaccaactttgatggccgcaaattaaagccttcga

SEQ ID NO: 23 - DNA, 55 bp, synthetic construct
  source 1..55: mol_type = other DNA
ctcgaaggctttaatttgcggccatcaaagttggtaatccatttgacaaggctaa

SEQ ID NO: 24 - DNA, 38 bp, synthetic construct
  source 1..38: mol_type = other DNA
ttacaacttaattctgacagcttttacttcagtgtatg

SEQ ID NO: 25 - DNA, 31 bp, synthetic construct
  source 1..31: mol_type = other DNA
gttaccattgcaatcaactgtctaagagatg

SEQ ID NO: 26 - DNA, 27 bp, synthetic construct
  source 1..27: mol_type = other DNA
caagaacgaattccctacattgaaggt

SEQ ID NO: 27 - DNA, 20 bp, synthetic construct
  source 1..20: mol_type = other DNA
caagtcgaccttggcactgg

SEQ ID NO: 28 - DNA, 35 bp, synthetic construct
  source 1..35: mol_type = other DNA
gccatgtaatatgattattaaacttctttgcgtcc

SEQ ID NO: 29 - DNA, 47 bp, synthetic construct
  source 1..47: mol_type = other DNA
gttaatatacctctatactttaacgtcaaggagaaaaaactataatg

SEQ ID NO: 30 - DNA, 27 bp, synthetic construct
  source 1..27: mol_type = other DNA
gaaaacggttggtctgatgaagtaacc

SEQ ID NO: 31 - DNA, 28 bp, synthetic construct
  source 1..28: mol_type = other DNA
atgtctctcctgcatcactaaatgtgtt

SEQ ID NO: 32 - DNA, 55 bp, synthetic construct
  source 1..55: mol_type = other DNA
gtaatcatggtcatagctgtttcctgtcagcaaagtcaagaatccaaattctggc

SEQ ID NO: 33 - DNA, 52 bp, synthetic construct
  source 1..52: mol_type = other DNA
gaatttggattcttgactttgctgacaggaaacagctatgaccatgattacg

SEQ ID NO: 34 - DNA, 45 bp, synthetic construct
  source 1..45: mol_type = other DNA
ggagtagaaacattttgaagctattaaaacgacggccagtgccaa

SEQ ID NO: 35 - DNA, 56 bp, synthetic construct
  source 1..56: mol_type = other DNA
gcactggccgtcgttttaatagcttcaaaatgtttctactccttttttactcttcc

SEQ ID NO: 36 - DNA, 58 bp, synthetic construct
  source 1..58: mol_type = other DNA
gtttgatgttgattacacataaacttagattagattgctatgctttctttctaatgag

SEQ ID NO: 37 - DNA, 54 bp, synthetic construct
  source 1..54: mol_type = other DNA
catagcaatctaatctaagtttatgtgtaatcaacatcaaacccgtgtattaag

SEQ ID NO: 38 - DNA, 57 bp, synthetic construct
  source 1..57: mol_type = other DNA
gtgacataactaattacatgattacaatgtggaaatatcaatcacaaatcgataacg

SEQ ID NO: 39 - DNA, 58 bp, synthetic construct
  source 1..58: mol_type = other DNA
gtgattgatatttccacattgtaatcatgtaattagttatgtcacgcttacattcacg

SEQ ID NO: 40 - DNA, 41 bp, synthetic construct
  source 1..41: mol_type = other DNA
gagtgtttatgggtgtcatggccgcaaattaaagccttcga

SEQ ID NO: 41 - DNA, 44 bp, synthetic construct
  source 1..44: mol_type = other DNA
gaaggctttaatttgcggccatgacacccataaacactcccgac

SEQ ID NO: 42 - DNA, 31 bp, synthetic construct
  source 1..31: mol_type = other DNA
acaataccatataccaacggcaatattcagc

SEQ ID NO: 43 - DNA, 31 bp, synthetic construct
  source 1..31: mol_type = other DNA
gatttccttccaggatgtatcttagacgaac

SEQ ID NO: 44 - DNA, 25 bp, synthetic construct
  source 1..25: mol_type = other DNA
gaacaatacaccgttccagaagtgc

SEQ ID NO: 45 - DNA, 31 bp, synthetic construct
  source 1..31: mol_type = other DNA
ctcgatgtagatgcctatttatcaatgcttc

SEQ ID NO: 46 - DNA, 40 bp, synthetic construct
  source 1..40: mol_type = other DNA
gtagctttaggattcatgaatattgtcatctcattattcg

SEQ ID NO: 47 - DNA, 27 bp, synthetic construct
  source 1..27: mol_type = other DNA
cagaaaaacagatgtgcccaaatccac

SEQ ID NO: 48 - DNA, 53 bp, synthetic construct
  source 1..53: mol_type = other DNA
catggtcatagctgtttcctggatcctaaactgcgtcatagtaagtttctttg

SEQ ID NO: 49 - DNA, 51 bp, synthetic construct
  source 1..51: mol_type = other DNA
cttactatgacgcagtttaggatccaggaaacagctatgaccatgattacg

SEQ ID NO: 50 - DNA, 46 bp, synthetic construct
  source 1..46: mol_type = other DNA
gcgacacggaaatgttgaatactcattaaaacgacggccagtgcca

SEQ ID NO: 51 - DNA, 48 bp, synthetic construct
  source 1..48: mol_type = other DNA
cttggcactggccgtcgttttaatgagtattcaacatttccgtgtcgc

SEQ ID NO: 52 - DNA, 46 bp, synthetic construct
  source 1..46: mol_type = other DNA
caatagtatgaataatttgcatccaatgcttaatcagtgaggcacc

SEQ ID NO: 53 - DNA, 58 bp, synthetic construct
  source 1..58: mol_type = other DNA
cctcactgattaagcattggatgcaaattattcatactattgaagaattgagacaagc

SEQ ID NO: 54 - DNA, 53 bp, synthetic construct
  source 1..53: mol_type = other DNA
gcaagatttaaagtaaattcactttaagcagctctttcttgagtaatttgtgg

SEQ ID NO: 55 - DNA, 60 bp, synthetic construct
  source 1..60: mol_type = other DNA
gagctgcttaaagtgaatttactttaaatcttgcatttaaataaattttctttttatagc

SEQ ID NO: 56 - DNA, 51 bp, synthetic construct
  source 1..51: mol_type = other DNA
gctttacgatggagtagtagacctaagggaaagatatgagctatacagcgg

SEQ ID NO: 57 - DNA, 51 bp, synthetic construct
  source 1..51: mol_type = other DNA
cgctgtatagctcatatctttcccttaggtctactactccatcgtaaagcc

SEQ ID NO: 58 - DNA, 41 bp, synthetic construct
  source 1..41: mol_type = other DNA
tgaggaatttacaataaggtggttcctttagttataaattg

SEQ ID NO: 59 - DNA, 46 bp, synthetic construct
  source 1..46: mol_type = other DNA
ctatttataaacgtcactaactagaaatacgggatatcaactacta

SEQ ID NO: 60 - DNA, 24 bp, synthetic construct
  source 1..24: mol_type = other DNA
caaaaacaggaaggcaaaatgccg

SEQ ID NO: 61 - DNA, 26 bp, synthetic construct
  source 1..26: mol_type = other DNA
gtaagccctcccgtatcgtagttatc

SEQ ID NO: 62 - DNA, 31 bp, synthetic construct
  source 1..31: mol_type = other DNA
gagaactgaaatttccataccctttaaccct

SEQ ID NO: 63 - DNA, 30 bp, synthetic construct
  source 1..30: mol_type = other DNA
caattcaagatacagaacctcctccagatg

SEQ ID NO: 64 - DNA, 53 bp, synthetic construct
  source 1..53: mol_type = other DNA
cgtaatcatggtcatagctgtttcctggtggaaagaacgggaaaccaactatc

SEQ ID NO: 65 - DNA, 54 bp, synthetic construct
  source 1..54: mol_type = other DNA
gatagttggtttcccgttctttccaccaggaaacagctatgaccatgattacgc

SEQ ID NO: 66 - DNA, 38 bp, synthetic construct
  source 1..38: mol_type = other DNA
gaaacattttgaagctattaaaacgacggccagtgcca

SEQ ID NO: 67 - DNA, 55 bp, synthetic construct
  source 1..55: mol_type = other DNA
gcactggccgtcgttttaatagcttcaaaatgtttctactccttttttactcttc

SEQ ID NO: 68 - DNA, 44 bp, synthetic construct
  source 1..44: mol_type = other DNA
cactgggggccaagcacagggggccgcaaattaaagccttcgag

SEQ ID NO: 69 - DNA, 39 bp, synthetic construct
  source 1..39: mol_type = other DNA
ctcgaaggctttaatttgcggccccctgtgcttggcccc

SEQ ID NO: 70 - DNA, 36 bp, synthetic construct
  source 1..36: mol_type = other DNA
gccgatttgtattagtatccaaattatgattccatg

SEQ ID NO: 71 - DNA, 27 bp, synthetic construct
  source 1..27: mol_type = other DNA
cttccatagagagaaggagcaagcaac

SEQ ID NO: 72 - DNA, 32 bp, synthetic construct
  source 1..32: mol_type = other DNA
gtactagaggaggccaagagtaatagaaaaag

SEQ ID NO: 73 - DNA, 29 bp, synthetic construct
  source 1..29: mol_type = other DNA
cacttattacggaattggaatgagcacag

SEQ ID NO: 74 - DNA, 28 bp, synthetic construct
  source 1..28: mol_type = other DNA
cagcaaaacatgcttattgtaattgggc

SEQ ID NO: 75 - DNA, 1503 bp, Saccharomyces cerevisiae
  source 1..1503: mol_type = genomic DNA
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 76 - DNA, 1308 bp, Saccharomyces cerevisiae
  source 1..1308: mol_type = genomic DNA
atggactacaacaagagatcttcggtctcaaccgtgcctaatgcagctcccataagagtcggattcgtcggtctcaacgcagccaaaggatgggcaatcaagacacattaccccgccatactgcaactatcgtcacaatttcaaatcactgccttatacagtccaaaaattgagacttctattgccaccattcagcgtctaaaattgagtaatgccactgcttttcccactttagagtcatttgcatcatcttccactatagatatgatagtgatagctatccaagtggccagccattatgaagttgttatgcctctcttggaattctccaaaaataatccgaacctcaagtatcttttcgtagaatgggcccttgcatgttcactagatcaagccgaatccatttataaggctgctgctgaacgtggggttcaaaccatcatctctttacaaggtcgtaaatcaccatatattttgagagcaaaagaattaatatctcaaggctatatcggcgacattaattcgatcgagattgctggaaatggcggttggtacggctacgaaaggcctgttaaatcaccaaaatacatctatgaaatcgggaacggtgtagatctggtaaccacaacatttggtcacacaatcgatattttacaatacatgacaagttcgtacttttccaggataaatgcaatggttttcaataatattccagagcaagagctgatagatgagcgtggtaaccgattgggccagcgagtcccaaagacagtaccggatcatcttttattccaaggcacattgttaaatggcaatgttccagtgtcatgcagtttcaaaggtggcaaacctaccaaaaaatttaccaaaaatttggtcattgacattcacggtaccaagggagatttgaaacttgaaggcgatgccggcttcgcagaaatttcaaatctggtcctttactacagtggaactagagcaaacgacttcccgctagccaatggacaacaagctcctttagacccggggtatgatgcaggtaaagaaatcatggaagtatatcatttacgaaattataatgccattgtgggtaatattcatcgactgtatcaatctatctctgacttccacttcaatacaaagaaaattcctgaattaccctcacaatttgtaatgcaaggtttcgatttcgaaggctttcccaccttgatggatgctctgatattacacaggttaatcgagagcgtttataaaagtaacatgatgggctccacattaaacgttagcaatatctcgcattatagtttataa